(19) United States
(12) Patent Application Publication
(10) Pub. No.: US 2006/0276786 A1
(43) Pub. Date: Dec. 7, 2006

(54) Apparatus for accurately positioning fractured bone fragments toward facilitating use of an external ring fixator system

(76) Inventor: Mark R. Brinker, Houston, TX (US)

Correspondence Address: GREENBERG TRAURIG, LLP, 77 WEST WACKER DRIVE, SUITE 2500, CHICAGO, IL, 60601-1732, US

(21) Appl. No.: 11/440,517

(22) Filed: May 25, 2006

Related U.S. Application Data
(60) Provisional application No. 60/684,350, filed on May 25, 2005.

(51) Int. Cl. A61B 17/00

(52) U.S. Cl. 606/54

(57) ABSTRACT

An alignment jack apparatus is disclosed for use in association with a circular external ring fixator system and facilitating alignment of displaced fracture bone fragments. The jack apparatus comprise a body housing a position control mechanism, an attachment clip connected to the body for releasably retaining the jack apparatus upon an external ring such that the alignment jack apparatus may be positioned between the external ring and the exterior facing surface of an underlying limb. The position control mechanism extends a moveable shaft from the body housing or retracts the shaft into the body housing such that a pressure plate connected to the shaft contacts the soft tissue of the limb and transmits a force to the underlying bone whereby an underlying bone fragment is repositioned as the pressure plate applies force to the external surface of the limb.

This application claims priority of the filing date of U.S. provisional patent application 60/684,350, filed 25 May 2005, the complete disclosure of which is hereby expressly incorporated herein by reference.

### BACKGROUND OF THE INVENTION

1. Field of the Invention

The present invention relates to aligning displaced fractured bone fragments using an external ring fixator system and, in particular, to an apparatus providing for the quick and highly accurate alignment of fractured bone segments toward facilitating placing and securing wires and pins to an external ring fixator.

2. Background and the Prior Art

Various methods are currently available and are widely used by orthopedic surgeons to reduce displaced fractured bone fragments. Various of these prior art methods utilize external fixation devices. The currently practiced methods using external ring fixators are technically demanding and often imprecise.

A typical prior art method for treating a displaced fracture utilizes an external fixator system which incorporates a plurality of rings, which together with supporting rods, form a framework surrounding a broken limb. The external ring fixator must be tailored to each individual patient and is preferably positioned so as to overlie the point of fracture. The rings are spaced apart and held together by a series of threaded rods which form the framework around the limb. The uppermost ring and lowermost rings each support a tensioned wire which is passed through the skin and underlying bone. These wires function to effectively secure the external ring framework to the upper and lower fractured bone fragments.

Once secured, the surgeon is able to reduce the fracture and align the broken bones. To reduce a fracture and align a broken bone a surgeon typically passes Olive Wires through the skin and bone at positions above and below the point of the fracture. Olive Wires are used to pull facture fragments into alignment in order to correct frontal plane angular deformities. The arched wire technique is often used to correct sagital plane angular and translational deformities.

An aperture is formed and the Olive Wire is fed through the skin and bone. An Olive Wire includes a deformation or protruding surface along its length which does not pass through the passage formed in the bone. The surgeon next manually pulls on the wire using a wire tensioner or puller, which typically comprises a lever style tool that grabs and pulls on the wire causing the bone to move in the direction the wire is pulled. Once in position, the wire is secured to a fixed position at a point along an external ring using a wire post or bolt. Thereafter the free end of the wire is tensioned using a wire tensioner or puller and affixed to the external ring using a wire post or bolt. It is often a cumbersome procedure to tension the wire and maintain the desired tension and position while securing the free ends of the wire to the ring.

In both techniques the fractured bone fragments are moved into a desired aligned positioned with respect to one another by the force exerted on the bone by the wire. For example, the deformation in the Olive Wire cannot pass through the bone such that pulling on the wire pulls on bone causing it to move. Alternatively, pulling on an arch wire draws it tight and thus shortens the wire which, in turn, moves the bone. The surgeon's skill and experience determines where along the ring the wire should be affixed and how tight the wire needs to be drawn in order to move the bone fragment to a desired position.

Once the wire is positioned and secured to the ring, an x-ray is taken and the position of the fractured bone fragments verified. The process is then repeated, sometimes again and again, until the desired position is achieved. The process is very tedious and time consuming. In fact, one significant disadvantage with this prior art method is that each successive step tends to displace the prior reduction with the surgeon adjusting one offset after another often never getting the fracture accurately reduced. In addition, oblique plane deformities which are neither purely in the frontal nor sagital plane are typically reduced by using these techniques in sequence, first reducing the fracture in the frontal plane and then reducing the fracture in the sagital plane.

Moreover, each adjustment requires that one or both ends of the wire be freed from its post or bolt such that tension on the wire is effectively released, notwithstanding the surgeon's attempt to maintain tension while making an adjustment. Accordingly this prior art method is generally a gross reduction maneuver that lacks the necessary precision and accuracy to optimally correct a displaced fracture.

Accordingly it is an object of the invention to provide for the precise reduction of a bone fracture and alignment prior to inserting wires or pins.

It is a further object of the present invention to provide for the precise reduction of a bone fracture in a quick and efficient manner which omits trial and error.

These and other objects of the present invention will become apparent to one of skill in the art having the present disclosure before them.

### SUMMARY OF THE INVENTION

An apparatus comprising an alignment jack for use in association with a circular external ring fixator system is disclosed for facilitating alignment of displaced fracture bone fragments. In a preferred embodiment the jack apparatus includes a body housing a position control mechanism actuated by a control device, such as a hexagonal knob. A moveable shaft is operably connected to the position control mechanism wherein the position control mechanism serves to alternatively extend the shaft from the body housing or retract the shaft into the body housing. A pressure plate is connected to the shaft for contacting soft tissue of the limb and transmitting a force to the underlying bone. An attachment clip is connected to the body housing for temporarily and releasably retaining the jack apparatus upon an external ring such that the alignment jack apparatus may be positioned between the external ring and the exterior facing surface of an underlying limb. Accordingly, an underlying bone fragment is able to be repositioned as the pressure plate applies force to the external surface of the limb.

In one embodiment of the present invention, the position control mechanism comprises a mechanical ball and screw. In other embodiments the position control mechanism may comprises a rack and pinion, an electronically controlled servo motor, a hydraulic motor or a pneumatic motor. The control device may alternatively comprise a recessed bolt or an external motor driven actuator.

In a preferred embodiment of the present invention, the pressure plate further includes padding to protect the skin surface of the underlying limb and the ring clip comprises a U-shaped flange suitable for engaging with an external fixator ring. In alternative embodiments, the ring clip may include a locking mechanism to releasably secure the clip onto the external fixator ring. It may also be adjustable with respect to the body housing so as to permit the angle of the moveable shaft to be altered with respect to the external fixator ring. In an alternative embodiment of the present invention the pressure plate may be replaced by a partial pin or a spike that directly contacts the bone fragment through an incision made in the skin.

A still further embodiment of the present invention is disclosed as comprising an automated computer assisted system for aligning displaced fractured bone fragments. Such a system is disclosed as comprising an external ring fixator structure including at least four external fixator rings affixed to one another by a plurality of rods and bolts permitting the rings to be positioned with at least a first ring above the point fracture and a second ring below the point of fracture and secured to the upper and lower fractured bone fragments with tensioned wires passed through the skin and underlying bone. A plurality of alignment jacks are removeably secured to at least the first and second external fixator rings toward facilitating alignment of the displaced fracture bone fragments. A computer controlled electronic servo motor is operably connected to each alignment jack for precisely and independently adjusting the position of each alignment jack. Computer control modules are electronically connected to each electronic servo motor for controlling the position of each adjustment jack. A computer controlled x-ray imaging and control system is provided for creating and analyzing an electronic image. The imaging and control system determines the relative displacement of the fractured bone fragments and signals the computer control modules to adjust the position of each alignment jack toward aligning the displaced fractured bone fragments without manual intervention. Reference wires capable may be inserted into the fractured bone fragments to enhance detection of the bone fragments by the x-ray imaging and control system and establish the initial position of the fractured bone fragments.

The computer controlled x-ray imaging and control system may also be configured to analyze the initial position of the fractured bone fragments, compute the distance and sequence in which each bone fragment must be moved in order to properly restore alignment and signal the computer control modules in a coordinated manner to move in the proper sequence toward articulating the fractured bone fragments into proper alignment. Back pressure sensors associated with one or more alignment jacks may further be provided to monitor the force exerted by the jack upon the limb and provide an alarm signal to the x-ray imaging and control system.

A method for aligning displaced fractured bone fragments is also disclosed as comprising the steps of: affixing an external ring fixator system to the fractured limb with at least one ring positioned above the fracture and one ring below the fracture; affixing an alignment jack to a ring positioned above and below the fracture; adjusting each alignment jack to reduce the fracture and align the bone fragments; inserting wires to retain the bone fragments in a stable position and securing the wires to the rings; and removing the alignment jacks. The fracture is thereby reduced and bone fragments aligned without the surgeon having to overcome forces otherwise exerted by traditional wires and the misaligned bone fragments, further permitting the surgeon to secure the fractured bones to the external rings while the bone fragments are in a stable condition and not susceptible to undesired movement.

### BRIEF DESCRIPTION OF THE DRAWINGS

FIG. 1 of the drawings illustrates a front elevation view of a human leg depicting a fracture of the tibia and fibula bones with a translational deformity;

FIG. 2 of the drawings illustrates a left side elevation view of a human leg depicting a fracture of the tibia and fibula bones with apex posterior angulation;

FIG. 3 of the drawings illustrates a front elevation view of a human leg depicting use of an external ring fixator system and the present innovative alignment jack apparatus toward reducing a fracture with translational deformity and aligning the tibia and fibula bones;

FIG. 4 of the drawings illustrates a left side elevation view of a human leg depicting use of an external ring fixator system and the present innovative alignment jack toward reducing a fracture with apex posterior angulation and aligning the tibia and fibula bones;

FIG. 5 of the drawings illustrates a front elevation view of a human leg depicting use of an external ring fixator system and the present innovative alignment jack wherein a fracture with translational deformity has been reduced and the tibia and fibula bones are aligned;

FIG. 6 of the drawings illustrates a left side elevation view of a human leg depicting use of an external ring fixator system and the present innovative alignment jack apparatus wherein a fracture with apex posterior angulation has been reduced and the tibia and fibula bones are aligned;

FIG. 7 of the drawings illustrates a top plan view of the alignment jack of the present invention;

FIG. 8 of the drawings illustrates a right side elevation view of the alignment jack of the present invention;

FIG. 9 of the drawings illustrates a front elevation view of the alignment jack of the present invention;

FIG. 10 of the drawings illustrates an orthographic view of the alignment jack of the present invention;

FIG. 11 of the drawings illustrates a top cross-section view of a human limb and a portion of an external fixator ring bearing the alignment jack of the present invention applying pressure to the exterior of the limb toward biasing the bones into alignment; and

FIG. 12 of the drawings illustrates a further embodiment of the present invention comprising an automated computer assisted system for aligning displaced fractured bone fragments.

### DETAILED DESCRIPTION OF THE DRAWINGS

While this invention is susceptible of embodiment in many different forms, there are shown in the drawing and will be described in detail, several specific embodiments, with the understanding that the present disclosure is to be considered as an exemplification of the principles of the present invention and is not intended to limit the invention to the embodiments illustrated.

The present invention is illustrated in the context of fractured leg bones with the understanding the present invention has application in many other situations. For example, the present invention may be used to move and align deformities in long bones, joints and other bone structures.

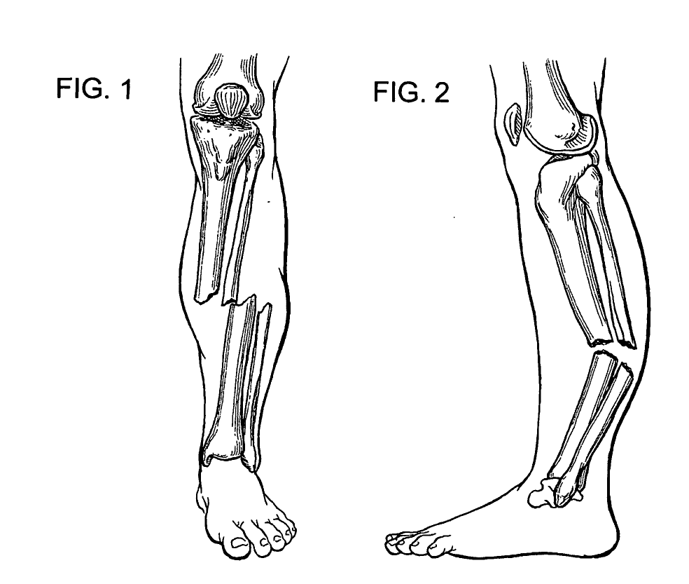

FIG. 1 of the drawings illustrates a front elevation view of a human leg depicting a fracture of the tibia and fibula bones with translational deformity. As illustrated, the bone above the point of the fracture is medially offset (to the left) while the bone below the point of the fracture is laterally offset (to the right), though both are generally parallel. To reduce the fracture and align the upper and lower fracture fragments, the upper fragment must be repositioned laterally (to the right) and the lower fragment repositioned medially (to the left).

FIG. 2 of the drawings illustrates a left side elevation view of a human leg depicting a fracture of the tibia and fibula bones with apex posterior angulation. In this illustration the bone fragments are not parallel. To reduce the fracture both the upper and lower fragments must be repositioned toward the front of the leg.

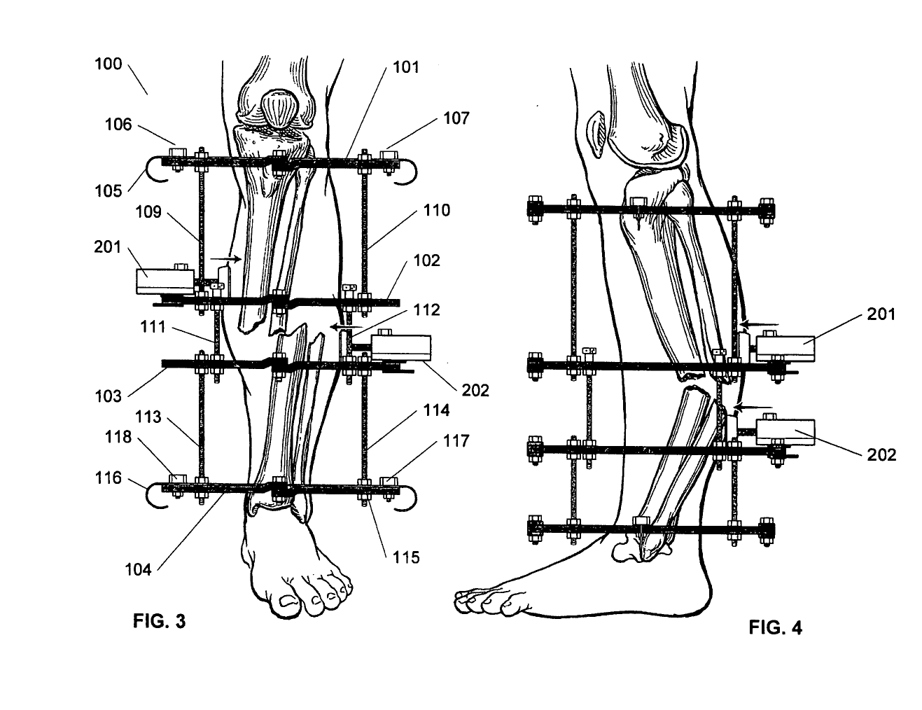

FIG. 3 of the drawings illustrates a front elevation view of a human leg depicting use of an external ring fixator system and the present innovative apparatus toward reducing a fracture with translational deformity and aligning the tibia and fibula bones. External ring fixator system 100 is illustrated comprising four rings 101-104, each of which are formed from two half rings joined together with connector bolts. Rings 101-104 are spaced apart by threaded rods 109-114 and affixed to the rings with bolts. The combination of rods and bolts permit the rings to be positioned as desired. While four rings appear in the illustrated example, the number of rings may vary depending upon the nature of the injury. Additionally, rings 101-104 may be whole rings, half rings, five-eights rings or any segment of a ring or arc.

Uppermost ring 101 and lower most ring 104 are secured to the limb by wires 105 and 116 in the conventional manner. Wires 105 and 116 are passed through the skin, tissue and bone of the limb and affixed to the rings 101 and 104, respectively, by bolts 106, 107, 117 and 118. The framework assembly is thus maintained in a stable orientation.

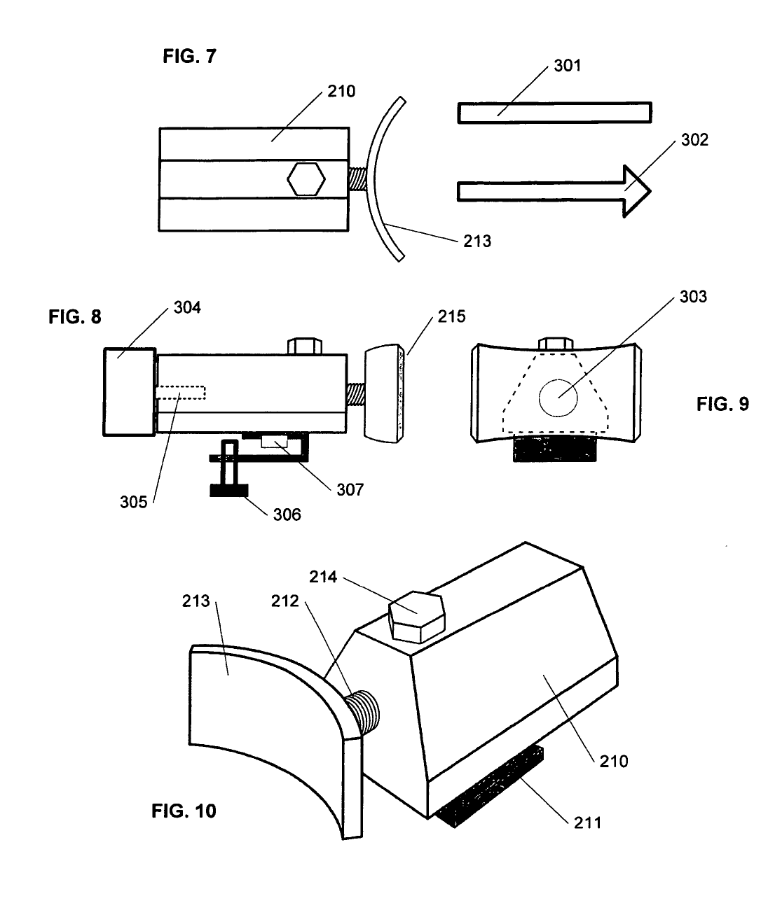

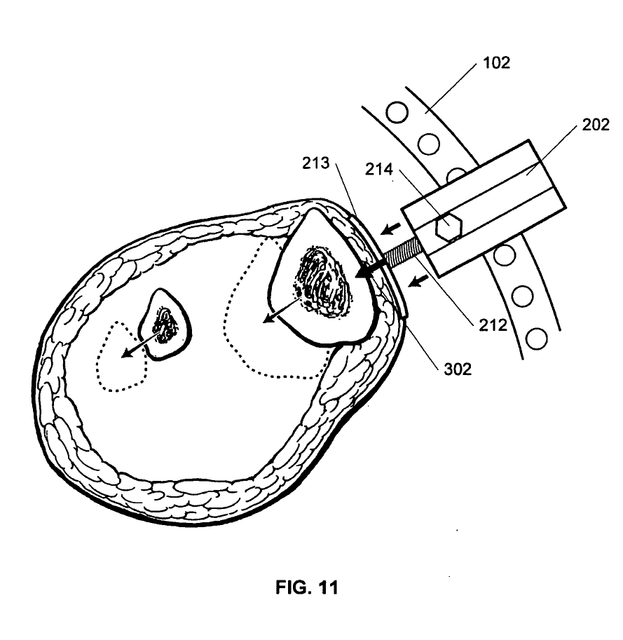

Alignment jacks 201 and 202 are shown releasably positioned upon rings 102 and 103, respectively. As illustrated in FIGS. 7-10, each alignment jack comprises a body 210 surrounding an adjustment mechanism, an extendable shaft 212, pressure plate 213 and ring clip 211 and adjustment bolt 214. Pressure plate 213 may include padding 215. Rotation of adjustment knob 214 serves to adjust the position of pressure plate 213 with respect to body 210 by extending or retracting shaft 212. One novel feature of present invention is the precise control offered by the micro-adjustment provided by the alignment jack. For example, it is contemplated that in one embodiment the adjustment mechanism within body 210 may comprise a mechanical ball and screw mechanism, with gear reduction, providing for precise control of movement of shaft 212 and, in turn, the position of pressure plate 213.

Alternative embodiments of the control mechanism are contemplated. For example, a rack and pinion mechanism may be used in place of a ball and screw structure. Additionally, motorized, hydraulic or pneumatic control devices are contemplated as being implemented to extend and retract shaft 212. For example, an electronically controlled precision servo motor 304 may be used to move shaft and, in turn, the position pressure plate 213.

Adjustment knob 214 is illustrated as comprising a hexagon shaped head affixed to an adjustment barrel (not shown). Knob 214 may be rotated by a socket or other similar tool. Alternate placement of knob 214 or another control mechanism is also contemplated. For example, an aperture 305 at the end of body 210 opposite pressure plate 213 may accept a tool or wrench to physically rotate shaft 212.

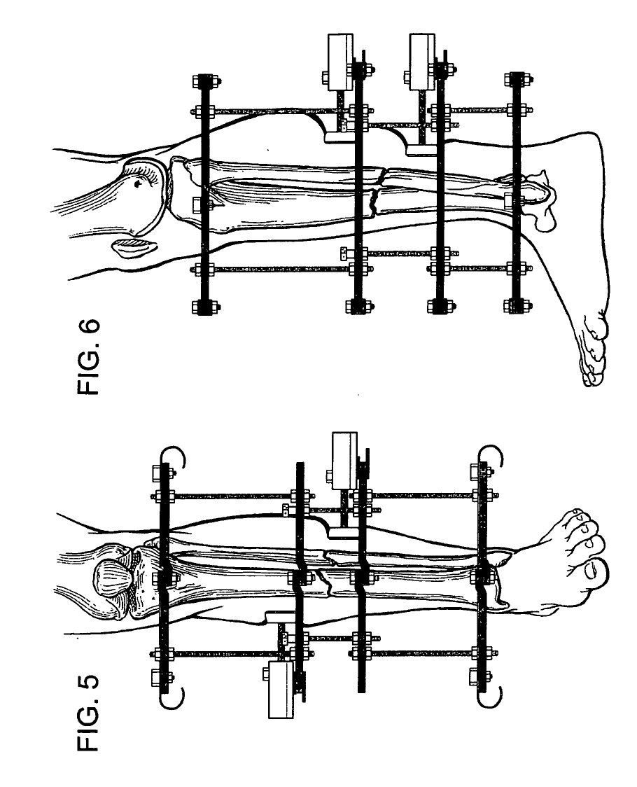

Ring clip 211 is illustrated as comprising a simple U-shaped flange suitable for engaging with an external fixator ring. Referring to FIG. 3, alignment jacks 201 and 202 are positioned on opposite sides of the limb. As shaft 212 rotates and extends pressure plate 213 of jack 201 ultimately contacts the exterior of the limb. As plate 213 advances further it applies pressure to the limb moving the proximal fragment (upper) laterally (to the right). As illustrated in FIG. 5, when properly extended, the forces exerted by jacks 201 and 202 cause a reduction in the fracture and alignment of the bones.

In practice it is contemplated that a surgeon will adjust jacks 201 and 202 to apply force to the limb to reposition the fracture fragments. A simple x-ray can be taken to verify the position of the fragments and further precise adjustments can be made to the jacks as needed to reduce the fracture and reposition the fragments into alignment.

As can be appreciated, changes to the position of pressure plate 213 can be made by simple adjustments to the jacks without having to release the pressure bearing on the fractured bone fragments in the process, unlike the prior art wherein the wire which must be released and repositioned each time an adjustment is desired. FIGS. 4 and 6 illustrate use of the present invention to reduce a fracture with apex posterior angulation wherein pressure is applied in the same direction above and below the point of the fracture. While two jacks and shown, more can be used with additional rings as the treatment warrants.

Once the surgeon is assured that the fracture is reduced and the bones aligned the surgeon may proceed to insert the necessary wires and/or pins to retain the bone fragments in their now stable position without having to overcome the forces which are otherwise exerted on the wire by the misaligned bone fragments. In short, the surgeon is able to secure the fractured bones to the external rings while the bone fragments are in a stable condition and not susceptible to undesired movement. When installation of the wires is complete, jacks 201 and 202 can be released and removed from rings 102 and 103.

Various embodiments and extensions of the present innovative apparatus are contemplated and deemed to be within the scope and spirit of the invention and disclosure.

For example, the alignment jacks can be constructed of a radiolucent material to facilitate and produce x-rays which are not obscured by fixation implements. The alignment jacks can be constructed of a MRI compatible material. Further, alignment jacks may be disposable.

While the alignment jacks as illustrated incorporate a shaft which extends outwardly and generally parallel to the longitudinal axis of body 210, shaft 212 may be configured to extend outwardly from body 210 at an angle toward altering the force vector applied to the bone fragment. Alternatively, clip 211 may be constructed to be adjustable via setting device 307 with respect to body 210 so as to alter the relative angle of shaft 212 and, in turn, the direction of force applied by pressure plate 213. Clip 211 may further include a locking mechanism 306 to secure the clip 211 to the ring 102 during use and may also be configured to conform to the shape or contour of various style rings. While pressure plate 213 and shaft 212 are shown as a generally unitary construction, a pivot assembly 303 can be provided between the distal end of shaft 212 and plate 213 to permit plate 213 to rotate. Pressure plate 213 can be fabricated in various shapes and/or lined with a variety of materials to prevent damage to the skin. In yet another embodiment, pressure plate 213 can be replaced by a partial pin 301 or spike 302 which directly contacts or penetrates the bone fragment though an incision made in the skin.

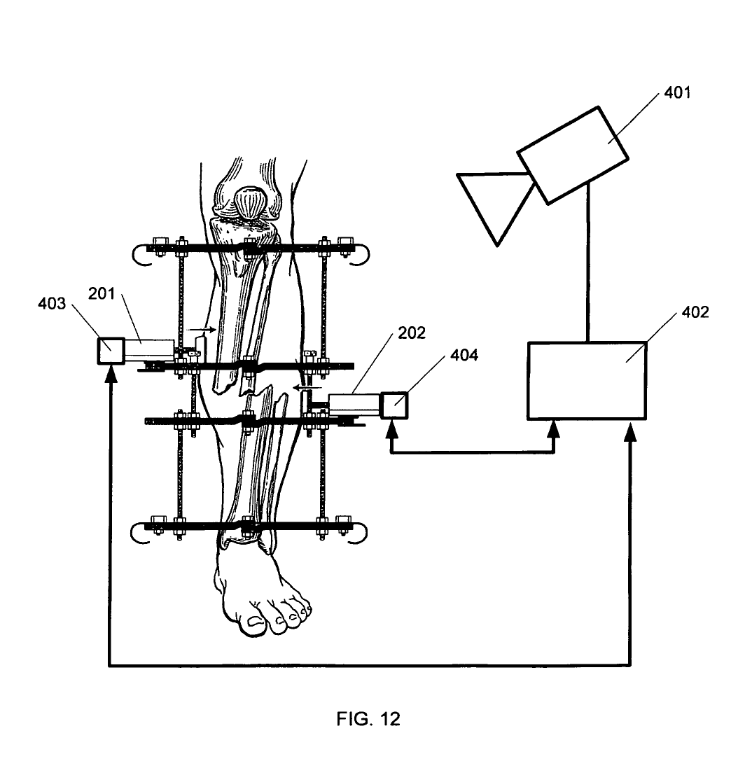

It is additionally contemplated that sophisticated control systems may be provided to synchronize the movement of a single or multiple adjustment jacks to provide uniform and/or automated control. For example as illustrated in FIG. 12, alignment jacks 201 and 202 are each connected to electronic servo motors 403 and 404, respectively, such that each jack is computer controlled and integrated into a fully automated alignment or stealth system. This system may optionally rely upon reference wires inserted by the surgeon to the distal and proximal bone fragments which when x-rayed by unit 401 and analyzed by the control computer 402 establish the initial position of the fractured bone fragments. A control computer 402 commands and controls the adjustment jacks 201 and 202 to advance the pressure plates (or pins or spikes) so as to, in turn, move the fractured bone fragments into alignment all without manual intervention. The system may further operate in an incremental manner where interim x-rays are taken and analyzed to monitor the progress and verify bone fragment position at various steps. Alternatively, the system may initially compute the total distance required to move the bone fragments into position to provide optimum alignment. The control system performs the mathematical computations necessary to determine the degree of movement. Option software analyzes the nature of the fracture whereby multiple alignment jacks may be moved in a coordinated manner to articulate bone fragments into proper alignment. Optional back pressure sensors are integrated into jacks 201 and 202 to monitor the force exerted on the limb and provide limits, warnings or otherwise permit optimum computer control over the movement of the individual bone fragments.

It can be further appreciated that the present invention permits a surgeon to work quicker and subject a patient to less trauma and the surgeon to less radiation.

The foregoing description and drawings merely explain and illustrate the invention and the invention is not limited thereto, as those skilled in the art who have the disclosure before them will be able to make modifications and variations therein without departing from the scope of the invention.

1. An alignment jack apparatus for use in association with a circular external ring fixator system and facilitating alignment of displaced fracture bone fragments, the jack apparatus comprising:
a body housing a position control mechanism;
an attachment clip operably connected to the body housing for temporarily and releasably retaining the jack apparatus upon an external ring such that the alignment jack apparatus may be positioned between the external ring and the exterior facing surface of an underlying limb;
a moveable shaft operably connected to the position control mechanism, the position control mechanism serving to alternatively extend the shaft from the body housing or retract the shaft into the body housing;
a control device for actuating the position control mechanism toward adjusting the position of the shaft; and
a pressure plate operably connected to the shaft for contacting soft tissue of the limb and transmitting a force to the underlying bone,
whereby an underlying bone fragment is repositioned as the pressure plate applies force to the external surface of the limb.

2. The alignment jack apparatus according to claim 1 wherein the position control mechanism comprises a mechanical ball and screw.

3. The alignment jack apparatus according to claim 1 wherein the position control mechanism comprises a rack and pinion.

4. The alignment jack apparatus according to claim 1 wherein the position control mechanism comprises an electronically controlled servo motor.

5. The alignment jack apparatus according to claim 1 wherein the position control mechanism comprises a hydraulic motor.

6. The alignment jack apparatus according to claim 1 wherein the position control mechanism comprises a pneumatic motor.

7. The alignment jack apparatus according to claim 1 wherein the control device comprises a knob.

8. The alignment jack apparatus according to claim 1 wherein the control device comprises a recessed bolt.

9. The alignment jack apparatus according to claim 1 wherein the control device comprises an external motor driven actuator.

10. The alignment jack apparatus according to claim 1 wherein the pressure plate further includes padding to protect the skin surface of the underlying limb.

11. The alignment jack apparatus according to claim 1 wherein the attachment clip comprises a U-shaped flange suitable for engaging with an external fixator ring.

12. The alignment jack apparatus according to claim 1 wherein the attachment clip includes a locking mechanism to releasably secure the clip on to the external fixator ring.

13. The alignment jack apparatus according to claim 1 wherein the attachment clip is adjustable with respect to the body housing so as to permit the angle of the moveable shaft to be altered with respect to the external fixator ring.

15. The invention according to claim 1 wherein the alignment jack apparatus is constructed of a radiolucent material to facilitate and product x-rays which are not obscured by fixation implements.

16. The invention according to claim 1 wherein the alignment jack apparatus is constructed of a MRI compatible material.

17. The alignment jack apparatus according to claim 1 wherein the moveable shaft extends outwardly and generally parallel to the longitudinal axis of the body housing.

18. The alignment jack apparatus according to claim 1 wherein the moveable shaft is connected to the pressure plate by a pivot assembly.

19. The alignment jack apparatus according to claim 1 wherein the pressure plate may be replaced by a partial pin with directly contacts the bone fragment through an incision made in the skin.

20. The alignment jack apparatus according to claim 1 wherein the pressure plate may be replaced by a spike with directly contacts the bone fragment through an incision made in the skin.

21. A system for aligning displaced fractured bone fragments, the system comprising:
an external ring fixator structure surrounding the broken limb including at least four external fixator rings affixed to one another by a plurality of rods and bolts permitting the rings to be positioned with at least a first ring above the point fracture and a second ring below the point of fracture and secured to the upper and lower fractured bone fragments with tensioned wires passed through the skin and underlying bone; and
a plurality of alignment jacks removeably secured to at least two external fixator rings facilitating alignment of the displaced fracture bone fragments.

22. The system according to claim 21 wherein the external fixator rings may be selected from a group of ring configurations comprising whole rings, half rings and five-eighth rings.

23. A automated computer assisted system for aligning displaced fractured bone fragments, the system comprising:
an external ring fixator structure including at least four external fixator rings affixed to one another by a plurality of rods and bolts permitting the rings to be positioned with at least a first ring above the point fracture and a second ring below the point of fracture and secured to the upper and lower fractured bone fragments with tensioned wires passed through the skin and underlying bone;
a plurality of alignment jacks removeably secured to at least the first and second external fixator rings toward facilitating alignment of the displaced fracture bone fragments;
a computer controlled electronic servo motor operably connected to each alignment jack for precisely and independently adjusting the position of each alignment jack;
computer controlled x-ray imaging and control system for creating and analyzing an electronic image, determining the relative displacement of the fractured bone fragments and signaling the controlled electronic servo motors to adjust the position of each alignment jack toward aligning the displaced fractured bone fragments without manual intervention.

24. The automated computer assisted system for aligning displaced fractured bone fragments according to claim 22 further including reference wires capable of being inserted into the fractured bone fragments to enhance detection of the bone fragments by the x-ray imaging and control system and establish the initial position of the fractured bone fragments.

25. The automated computer assisted system for aligning displaced fractured bone fragments according to claim 22 wherein computer controlled x-ray imaging and control system analyzes the initial position of the fractured bone fragments, computes the distance and sequence in which each bone fragment must be moved in order to properly restore alignment and signals the controlled electronic servo motors in a coordinated manner to move in the proper sequence toward articulating the fractured bone fragments into proper alignment.

26. The invention according to claim 22 wherein the automated computer assisted system further includes back pressure sensors associated with one or more alignment jacks toward monitoring the force exerted by the jack upon the limb and providing an alarm signal to the x-ray imaging and control system.

27. A method for aligning displaced fractured bone fragments, the method comprising the steps of:
affixing an external ring fixator system to the fractured limb with at least one ring positioned above the fracture and one ring below the fracture;
affixing an alignment jack to a ring positioned above and below the fracture;
adjusting each alignment jack to reduce the fracture and align the bone fragments;
inserting wires to retain the bone fragments in a stable position and securing the wires to the rings; and
removing the alignment jacks;
whereby the fracture is reduced and bone fragments aligned without the surgeon having to overcome forces otherwise exerted by traditional wires and the misaligned bone fragments, further permitting the surgeon to secure the fractured bones to the external rings while the bone fragments are in a stable condition and not susceptible to undesired movement.
